(19) United States
(12) Patent Application Publication
(10) Pub. No.: US 2008/0315127 A1
(43) Pub. Date: Dec. 25, 2008

(54) Ion Implanter Operating in Pulsed Plasma Mode

(76) Inventors: Frank Torregrosa, Simiane (FR); Gilles Mathieu, Marseille (FR); Laurent Roux, Marseille (FR)

Correspondence Address: Horst M. Kasper, 13 Forest Drive, Warren, NJ, 07059, US

(21) Appl. No.: 11/629,690

(22) PCT Filed: Jun. 14, 2005
(86) PCT No.: PCT/FR2005/001468

§ 371 Date: Mar. 12, 2007

(30) Foreign Application Priority Data
Jun. 16, 2004 (FR) 0406496

(51) Int. Cl. A61N 5/00

(52) U.S. Cl. 250/492.3

(57) ABSTRACT

The present invention relates to an ion implanter IMP comprising a pulsed plasma source SPL, a substrate-carrier tray PPS, and a power supply ALT for the tray. The implanter also includes a capacitor C connected directly to ground E and connected downstream from the tray power supply ALT. The invention also provides a method of using the implanter.

The present invention relates to an ion implanter operating in pulsed plasma mode.

The field of the invention is that of ion implanters operating in plasma immersion mode. Thus, implanting ions in a substrate consists in immersing the substrate in a plasma and in biasing it with a negative voltage, in the range a few tens of volts to a few tens of kilovolts (generally less than 100 kV), in order to create an electric field capable of accelerating ions from the plasma towards the substrate.

The depth to which ions penetrate is determined by their acceleration energy. It depends firstly on the voltage applied to the substrate and secondly on the respective natures of the ions and of the substrate. The concentration of implanted atoms depends on the dose that is expressed in terms of number of ions per square centimeter (cm²) and on the implantation depth.

For reasons associated with the physics of plasmas, a few nanoseconds after the voltage is applied, an ion sheath is created around the substrate. The potential difference responsible for accelerating ions towards the substrate is the difference to be found across this sheath.

The growth of this sheath as a function of time satisfies the Child-Langmuir law:

$${j}_{e} = \frac{4}{9} {{ɛ}_{0} ( \frac{2 e}{M} )}^{1 / 2} \frac{{V}_{0}^{3 / 2}}{{s}^{2}}$$

where:
j_{c}=current density;
e₀=permittivity of free space;
e=ion charge;
M=ion mass;
V₀=potential difference through the sheath; and
s=thickness of the sheath.

By stipulating that the current density is equal to the charge passing through the boundary of the sheath per unit time, ds/dt represents the displacement of said boundary:

$$\frac{d s}{d t} = \frac{2}{9} \frac{{s}_{0}^{2} \cdot {u}_{0}}{{s}^{2}}$$

In which the expression s₀ is given by:

$${s}_{0} = {( \frac{2 {ɛ}_{0} {V}_{0}}{e \cdot {n}_{0}} )}^{1 / 2}$$

it being understood that u₀=(2eV₀/M) is the characteristic speed of the ion and that n₀ is the density of the plasma.

The thickness of the sheath is associated mainly with the applied voltage, the density of the plasma, and the mass of the ions.

The equivalent impedance of the plasma, which conditions implantation current, is directly proportional to the square of the thickness of the sheath. Implantation current thus decreases very quickly when the sheath becomes larger.

After a certain amount of time has elapsed, it is necessary to reinitialize. In practice this is found to be essential when the sheath reaches the walls of the enclosure, thereby stopping the implantation mechanism.

In order to reinitialize the system, almost all implanter manufacturers disconnect the high voltage from the substrate while keeping the plasma ignited. It is therefore necessary to have a pulse generator that produces high-voltage pulses.

Furthermore, implantation requires acceleration energy to be as stable as possible, and consequently it is appropriate to satisfy the following specifications:
rise and fall times less than 1 microsecond (μs);
high voltage stable during the pulse;
instantaneous current very high in the range 1 amp (A) to 300 A; and
ability to accommodate arcing in the plasma.

Ion implantation in plasma immersion mode presents a certain number of drawbacks.

Firstly, pulsed high voltage power supplies are very expensive, often fragile, and have a direct influence on the quality of the implantation performed.

Secondly, the continuous presence of the plasma in the enclosure gives rise to undesirable side effects:
particle generation;
heat delivered to the substrate;
the enclosure is attacked, giving rise to a risk of metal contamination of the parts being processed; and
charge effects are created, which can be particularly troublesome in microelectronic applications.

In order to reduce those side effects, the supplier Varian has proposed a pulsed plasma process referred to as plasma doping (PLAD). That process is described in two articles of the journal Surface and Coatings Technology, No. 156 (2002) “Proceedings of the VIth international workshop on plasma-based ion implantation (PBII-2001), Grenoble, France, Jun. 25-28, 2001” published by Elsevier Science B.V.:
S. B. Felch et al., “Plasma doping for the fabrication of ultra-shallow junctions”, pp. 229-236; and
D. Lenoble et al., “The fabrication of advanced transistors with plasma doping”, pp. 262-266.

That method also consists in biasing the substrate with high voltage pulses. Nevertheless, the electric field created between the substrate and the ground electrode situated facing it enables the plasma to be pulsed. The field lines around the substrate enable ions to be accelerated and implanted. In that method, the pulsed plasma makes it possible to avoid some of the above-described side effects, but the constraints associated with using a high voltage pulsed generator still remain. Furthermore, the characteristic of the plasma cannot be separate from the bias voltage. As a result, the machine is not very versatile: it presents a small range of acceleration voltages and it is always difficult to implant species that do not lend themselves to forming plasmas.

Using a different approach, U.S. Pat. No. 5,558,718 teaches apparatus and a method for implanting ions having a source of pulses. That ion implantation apparatus does not have a high voltage pulse generator. It makes use of a pulsed plasma source and a constant voltage applied to the target by a power source. When large targets are used that require high currents, a high-capacitance circuit is connected in parallel with the power source. That circuit comprises a resistor and a capacitor in series and presents certain limitations.

Firstly, it consumes a large amount of energy. Secondly, it needs to be designed in a manner that matches the volume of the target to be ionized. Finally, the time constant of the parallel circuit must be longer than the duration of a pulse from the generator.

Mention can also be made to document DE 195 38 903 which proposes apparatus provided with a plasma source, a substrate-carrier tray, and a power supply for the tray. That apparatus has a resistor connected between the tray and the power supply; a capacitor connected to ground is connected to the common point between the power supply and the resistor. In addition to the limitations mentioned for the preceding document, the resistor is provided here in order to limit arcing current and generates a potential drop across its terminals. The magnitude of this potential drop depends on the magnitude of the implantation current and thus greatly disturbs control over the acceleration voltage that is applied to the substrate carrier.

The invention proposes providing an improvement to the above situation.

According to the invention, an ion implanter comprises a pulsed plasma source, a substrate-carrier tray, and a power supply connected directly between ground and the substrate-carrier tray; in addition, it includes a capacitor connected between ground and said substrate-carrier tray.

In a first embodiment, the tray power supply comprises a direct voltage source connected in series with a load impedance.

Under such circumstances, it includes means for ensuring that the duration of the plasma pulse emitted by the pulsed plasma source lies in the range 15 microseconds (μs) to 500 μs.

Preferably, the impedance is a resistance lying in the range 100 kilohms (kΩ) to 1000 kΩ.

Similarly, the capacitor has capacitance lying in the range 5 nanofarads (nF) to 5 microfarads (μF).

The invention also provides an implantation method implementing such an implanter, the method comprising periodically repeating at least the following four stages:
a stage of charging the capacitor from the voltage source until a discharge voltage is obtained;
a stage of igniting the plasma;
a stage of discharging the capacitor; and
after a predetermined delay, a stage of extinguishing the plasma.

In a second embodiment, the tray power supply is a direct current (DC) source.

Under such circumstances, the implanter includes means for ensuring that the duration of the plasma pulse emitted by the pulsed plasma source lies in the range 15 μs to 500 μs.

Advantageously, the capacitor has capacitance lying in the range 5 nF to 5 μF.

The implantation method corresponding to the second embodiment is identical to that defined above for the first embodiment.

In general, these methods provide for the plasma to be ignited for a duration lying in the range 1 μs to 10 milliseconds (ms).

In addition, after the extinction stage, these methods include a waiting stage.

Furthermore, the plasma presents density of 10⁸ ions per cubic centimeter (ions/cm³) to 10¹⁰ ions/cm³ for a working pressure of 2×10⁻⁴ millibars (mbar) to 5×10⁻³ mbar.

Currently, the voltage used for application to the tray lies in the range −50 V to −100 kV.

The frequency of the plasma pulses usually lies in the range 1 hertz (Hz) to 14 kilohertz (kHz).

According to an additional characteristic, the substrate-carrier tray is rotatable about its axis.

The substrate-carrier tray and the pulsed plasma source preferably have axes that are parallel with an adjustable offset.

The present invention is described below in greater detail in the following description of an embodiment given by way of illustration with reference to the accompanying figures, in which:

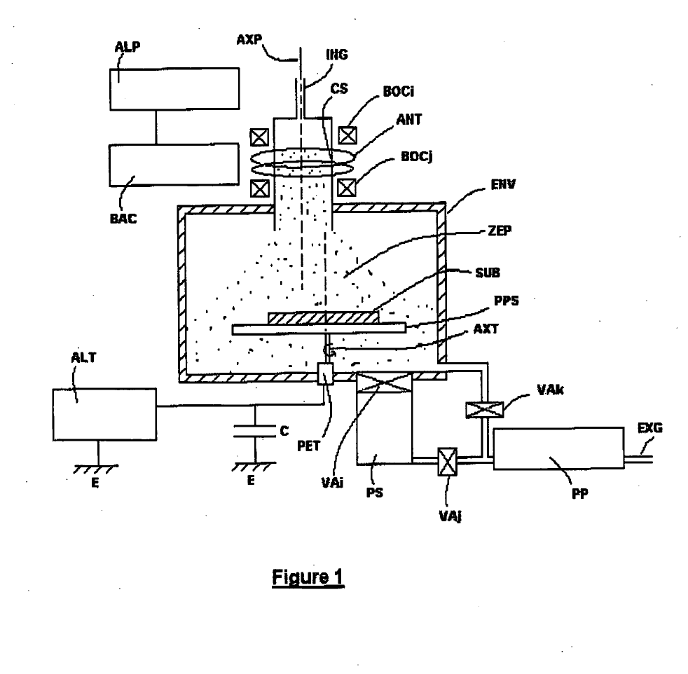

FIG. 1 shows an implanter in diagrammatic vertical section;

FIG. 2 shows a first variant power supply for the tray; and

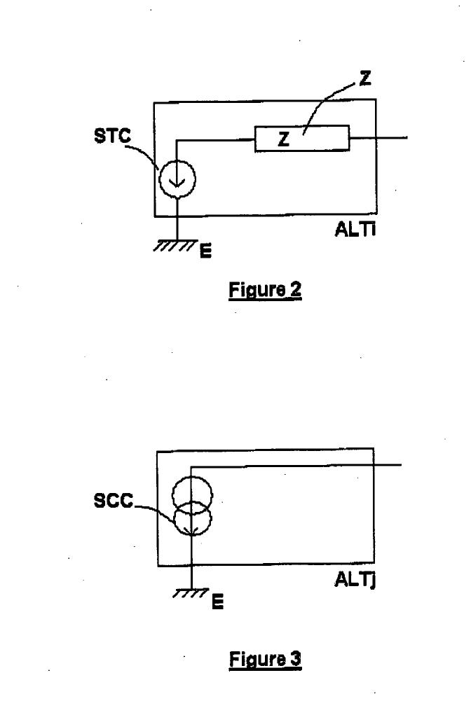

FIG. 3 shows a second variant power supply for the tray.

Elements that are present in more than one figure are given the same reference in all of them

As shown in FIG. 1, an ion implanter IMP comprises a plurality of elements arranged inside and outside a vacuum enclosure ENV. For microelectronic applications, it is recommended to use an enclosure made of aluminum alloy if it is desired to limit contamination with metallic elements such as iron, chromium, nickel, or cobalt. A coating of silicon or of silicon carbide may also be used.

A substrate-carrier tray PPS is provided in the form of a horizontal plane disk that is rotatable about its vertical axis AXT, and it receives the substrate SUB that is to have ions implanted therein.

A high voltage bushing PET formed through the bottom portion of the enclosure ENV provides an electrical connection between the tray vertical axis AXT, and thus the substrate-carrier tray PPS, and a tray power supply ALT that is in turn connected to ground E. A capacitor C also connected to ground E is connected downstream from the tray power supply ALT; in other words, the capacitor C is connected between the substrate-carrier tray PPS and ground E.

Pump means PP, PS are also disposed in the bottom portion of the enclosure ENV. A primary pump PP has its inlet connected to the enclosure ENV by a pipe provided with a valve VAk, and its outlet connected to the atmosphere via an exhaust pipe EXG. A secondary pump PS has its inlet connected to the enclosure ENV by a pipe provided with a valve VAi, and has its outlet connected to the inlet of the primary pump PP via a pipe provided with a valve VAj. The pipes themselves are not referenced.

The top portion of the enclosure ENV receives the source body CS that is cylindrical in shape about a vertical axis AXP. This body is made of quartz. It is surrounded externally firstly by confinement coils BOCi, BOCj, and secondly by an outer radiofrequency (RF) antenna ANT. The antenna is connected electrically via a tuning box BAC to a pulsed RF power supply ALP. The inlet ING for plasma-generating gas is coaxial about the vertical axis AXP of the source body CS. This vertical axis AXP intersects the surface of the substrate-carrier tray PPS on which the substrate SUB for implanting is placed.

It is possible to use any type of pulsed plasma source: discharge; inductively-coupled plasma (ICP); Helicon; microwaves; arc. The source must operate at a pressure level that is low enough for the electric field created between the tray PPS at high voltage and the enclosure ENV at ground potential does not ignite a discharge plasma which would disturb the pulsed operation of the source.

The selected source must be capable of having a plasma potential that is close to zero. The acceleration energy of the ions is the difference between the plasma potential and the substrate potential. The acceleration energy is thus controlled solely by the voltage applied to the substrate. This point becomes predominant if it is desired to have acceleration energies that are very low, less than 500 electron volts (eV), which is true for microelectronic applications.

For applications that require metallic contamination to be at a low level, such as microelectronics, as above, and also processing items for medical applications, the source must not present any contaminating metal element in contact with the plasma. In the embodiment described, an RF source formed by a quartz tube is associated with an external RF antenna ANT and with magnetic confinement coils BOCi, BOCj, as described above.

Three advantages of the FIG. 1 apparatus can be mentioned.

Firstly, the independence between the conditions required for igniting the plasma and the bias voltage of the substrate enable great versatility to be achieved in the range of energies that can be used.

Secondly, the possibility of using a very low bias voltage, e.g. less than 50 V or 100 V, constitutes an advantage for fabricating ultrafine junctions in electronic components,

Thirdly, the high voltage pulses are not present.

Any plasma-generating species can be implanted. This can be done from a gaseous precursor such as N₂, O₂, He, Ar, BF₃, B₂H₆, AsH₃, PH₃, SiH₄, C₂H₄, a liquid precursor such as TiCl₄, H₂O, or a solid precursor. With a solid precursor, it is appropriate to use a thermal evaporation system (phosphorus) or a hollow cathode arc system.

FIG. 2 shows a tray power supply module ALTi in a first embodiment of the invention. The tray power supply ALTi comprises a direct voltage source STC in series with a load impedance Z that is provided to limit current when beginning to charge the capacitor C. This load impedance is often a resistor. It may also be in the form of an inductor of inductance that depends on the capacitance of the capacitor C and on the impedance of the plasma.

Parameters that are commonly used in this embodiment are the following:
plasma density lying in the range 10⁸ ions/cm³ to 10¹⁰ ions/cm³;
plasma pulse duration lying in the range 15 μs to 500 μs;
pulse repetition frequency lying in the range 1 Hz to 3 kHz;
working pressure lying in the range 2×10⁻⁴ mbar to 5×10⁻³ mbar;
gas used: N₂, BF₃, O₂, H₂, PH₃, AsH₃, or Ar;
load impedance z constituted by a resistor having resistance of 330 kΩ±10%;
capacitor C having capacitance of 15 nF±10%; and
bias voltage lying in the range −100 V to −100 kV.

The implantation method implementing the implanter IMP comprises periodically repeating the following four or five stages:
a stage of charging the capacitor C (the plasma source SPL being extinguished) from the continuous voltage source STC through the load impedance Z until a discharge voltage is obtained;
a stage of igniting the plasma which is initiated when the voltage of the substrate reaches the discharge voltage: since the impedance of the plasma is no longer infinite, the capacitor C discharges through the plasma;
a stage of discharging the capacitor C, during which implantation is performed and during which the sheath becomes larger; and
a stage of extinguishing the plasma which is initiated when the preceding stage has lasted for a desired length of time: the impedance of the plasma is again infinite and the charging stage can be repeated; together with
an optional waiting stage during which nothing happens, thereby enabling the repetition period to be adjusted.

During the discharge stage, a plasma extension zone ZEP constituted by an ionized gas cloud forms between the source body CS and the substrate-carrier tray PPS. The particles strike the substrate SUB that is to be implanted with energy that enables them to penetrate into the substrate SUB.

FIG. 3 shows a preferred, second embodiment in which the tray power supply ALTj connected to ground E comprises a direct current source SCC.

The parameters commonly used in this embodiment are as follows:
plasma density lying in the range 10⁸ ions/cm³ to 10¹⁰ ions/cm³;
plasma pulse duration lying in the range 15 μs to 500 μs;
pulse repetition frequency lying in the range 1 Hz to 3 kHz;
working pressure lying in the range 5×10⁻⁴ mbar to 5×10⁻³ mbar;
gas used: BF₃, PH₃, AsH₃, N₂, O₂, H₂, or Ar;
capacitor C having capacitance of 1 μF; and
bias voltage lying in the range −100 V to −100 kV.

The implantation method implementing this embodiment of the implanter IMP is analogous to the method above, apart from the absence of the load impedance Z.

In this embodiment, a current source or capacitance charger is used directly and charging is stopped when the desired voltage is reached across the terminals of the capacitor. The advantage of this second embodiment is eliminating the load impedance Z which is an element that consumes power and constitutes a weakness of the machine.

On request, the primary and secondary pumps PP and PS achieve the desired level of vacuum inside the enclosure ENV after a substrate SUB has been placed on the substrate-carrier tray PPS.

The following parameters are generally adopted in both embodiments:
duration of plasma source ignition 1 μs to 1000 μs;
plasma density 10⁸ ions/cm³ to 10¹⁰ ions/cm³;
working pressure 2×10⁻⁴ mbar to 5×10⁻³ mbar;
bias voltage lying in the range −100 V to −100 kV;
plasma pulse frequency lying in the range 1 Hz to 14 kHz;
frequency of RF power supply 13.56 MHz±10%, in pulses.

The bias voltage may go from zero (no low voltage limit) to −100 kV. Greater voltages lead to significant risks of arcing.

The capacitance of the capacitor should be selected as a function of what it is desired to perform.

A large capacitance is needed to obtain a substrate voltage that is as stable as possible during the implantation stage. Thus, the amount of charge stored is much greater than the amount of charge consumed during the implantation stage.

A small capacitance enables the substrate voltage to drop during the implantation stage. Under such circumstances, the amount of charge stored is less than the amount of charge consumed during the implantation stage, thus assisting in extinguishing the plasma when working with a high substrate voltage at high pressure. Under such circumstances, there is a risk of auto-ignition by discharge between the tray and the walls of the enclosure.

The mean implantation current depends on the density of the plasma, on the bias voltage, and on the frequency and the duration of the plasma pulses. For stationary instantaneous conditions, the current can be set by adjusting the repetition period. For pulses at 50 keV, the current adjustment range is 1 microamps (μA) to 100 milliamps (mA). For implantation at 500 eV, the range is 1 μA to 10 mA.

The minimum value for the substrate voltage depends on the discharge time, equivalent to the plasma ignition time, and on the capacitance.

The maximum value of the substrate voltage depends on the charge of the capacitor.

The use of a capacitor of high capacitance makes it possible to obtain an acceleration voltage that is almost constant during the pulse. Under such circumstances, the product of multiplying the impedance of the plasma by the capacitance is much greater than the duration of the pulse.

An additional characteristic of the implanter shown in FIG. 1 makes it possible to obtain uniform implantation for a substrate of large size.

As mentioned above, the substrate SUB rests on a substrate-carrier tray PPS that is generally in the form of a disk and mounted to turn about its vertical axis AXT. With or without rotation, if the axis AXP of the plasma source SPL above the substrate SUB is close to the axis AXT of the tray PPS, then plasma diffusion is at a maximum along that axis and there will be a distribution gradient relative to the axis. The dose implanted in the substrate SUB will therefore present a distribution that is not uniform.

However, if the axes AXT and AXP are offset, then rotating the substrate-carrier tray PPS serves to move the substrate SUB relative to the axis AXP of the plasma source. The dose implanted in the substrate SUB will then present a distribution of uniformity that is considerably improved.

The effectiveness of this system has been verified on silicon wafers having a diameter of 200 mm for which the resulting non-uniformity was found to be less than 2.5% when implanting BF₃ at 500 eV and at 10¹⁵ ions/cm².

The embodiment of the invention described above was selected for its concrete nature. Nevertheless, it is not possible to list exhaustively all embodiments covered by the invention. In particular, any of the means described could be replaced by equivalent means without going beyond the ambit of the present invention.

1. An ion implanter IMP comprising a pulsed plasma source SPL, a substrate-carrier tray PPS, and a power supply ALTi, ALTj connected directly between the substrate-carrier tray and ground E, the implanter being characterized in that it includes a capacitor C connected between ground E and said substrate-carrier tray PP3.

2. An implanter IMP according to claim 1, characterized in that said power supply ALTi comprises a direct voltage source STC connected in series with a load impedance Z.

3. An implanter IMP according to claim 2, characterized in that it includes means for ensuring that the duration of the plasma pulse emitted by said pulsed plasma source SPL lies in the range 15 μs to 500 μs.

4. An implanter IMP according to claim 2, characterized in that said load impedance Z is a resistance lying in the range 100 k'Ω to 1000 'Ωk.

5. An implanter IMP according to claim 2, characterized in that said capacitor C has capacitance lying in the range 5 nF to 5 μF.

6. An implantation method implementing an implanter IMP according to claim 2, characterized in that it comprises periodically repeating at least the following four stages:
a stage of charging said capacitor C by said voltage source SPC to obtain a discharge voltalte;
a stage of igniting the plasma;
a stage of discharging said capacitor C; arid
after a predetermined delay, a stage of extinguishing the plasma.

7. An implanter IMP according to claim 1, characterized in that said tray power supply ALTj is a direct current source SCC.

8. An implanter IMP according to claim 7, characterized in that it includes means to ensure that the duration of the plasma pulse emitted by said pulse plasma source SPL lies in the range 15 μs to 500 μs.

9. An implanter IMP according to claim 7, characterized in that said capacitor C has capacitance lying in the range 5 nF to 5 μF.

10. An implantation method implementing the implanter IMP according to claim 7, characterized in that it comprises periodically repeating at least the following four stages:
a stage of charging said capacitor C by said voltage source STC to obtain a discharge voltalte;
a stage of igniting the plasma;
a stage of discharging said capacitor C; and
after a predetermined delay, a stage of extinguishing the plasma.

11. An implantation method according to claim 6, implementing an implanter IMP and characterized in that the duration of plasma source ignition lies in the range 1 μs to 10 ms.

12. An implantation method according to claim 6, characterized in that it includes a waiting stage following said extinction stage.

13. An implanter ZMP according to claim 1, characterized in that the plasma presents a density of 10⁸ ions/cm³ to 10¹⁰ ions/cm³ for a working pressure lying in the range 2×10−⁴ mbar to 5×10−³ mbar.

14. An implanter IMP according to claim 1, characterized in that the voltage used for powering the tray PPS lies in the range −50 V to −100 kV.

15. An implanter IMP according to claim 1, characterized in that the frequency of the plasma pulses lies in the range 1 Hz to 14 kHz.

16. An implanter IMP according to claim 1, characterized in that the substrate-carrier tray PPS is rotatable about its axis AXT.

17. An implanter IMP according to claim 16, characterized in that the substrate-carrier tray PPS of axis AXT and the pulsed plasma source SPL of axis AXP presents an adjustable offset.
